(19) United States
(12) Patent Application Publication
(10) Pub. No.: US 2025/0295897 A1
(43) Pub. Date: Sep. 25, 2025

(54) BRANCHING INTERNAL DRAIN AND EXTERNAL CIRCUIT

(71) Applicant: Northwestern University, Evanston, IL (US)

(72) Inventors: Ahsun RIAZ, Evanston, IL (US); Ali HUSNAIN, Evanston, IL (US); Heather MOLINA, Evanston, IL (US); James DONALDSON, Evanston, IL (US); Riad SALEM, Evanston, IL (US)

(21) Appl. No.: 19/082,620

(22) Filed: Mar. 18, 2025

Related U.S. Application Data
(60) Provisional application No. 63/567,139, filed on Mar. 19, 2024.

(51) Int. Cl. A61M 27/00; A61M 25/00; A61M 25/01; A61M 25/02; A61M 39/22

(52) U.S. Cl. CPC A61M 27/00; A61M 25/0028; A61M 25/007; A61M 25/0108; A61M 39/22; A61M 2025/0002; A61M 2025/0286; A61M 2039/229

(57) ABSTRACT

Provided herein are systems and methods concerning percutaneous drains that allow for improved fluid flow and patient comfort. In particular, the systems herein comprise a branching internal drain and a modified external circuit configuration by which a single drain insertion site can be used to place a branching internal drain through the first drain into the contralateral liver lobe/other liver segments to promote drainage from other sites, place a branching internal drain parallel to the first drain to increase the inner diameter of the drain across the stricture (ureteric or biliary), and place a branching internal drain into a large abscess to promote drainage from multiple areas or loculations within the abscess.

### CROSS REFERENCE TO RELATED APPLICATIONS

This application claims priority to and the benefit of U.S. Provisional Patent Application No. 63/567,139, filed Mar. 19, 2024, which is incorporated herein by reference in its entirety and for all purposes.

### FIELD

Provided herein are systems and methods concerning percutaneous drains that allow for improved fluid flow and patient comfort. In particular, the systems herein comprise a branching internal drain and a modified external circuit configuration by which a single drain insertion site can be used to place a branching internal drain through the first drain into the contralateral liver lobe/other liver segments to promote drainage from other sites, place a branching internal drain parallel to the first drain to increase the inner diameter of the drain across the stricture (ureteric or biliary), and place a branching internal drain into a large abscess to promote drainage from multiple areas or loculations within the abscess.

### BACKGROUND

Percutaneous drains are often placed in the biliary tract, urinary tract, and abscesses for various indications during interventional radiology procedures. Percutaneous biliary drains have high complication rates, particularly in patients with non-dilated ducts. Such complications may include pain, leakage, and catheter obstruction, and percutaneous puncturing of the ducts can cause vascular injury and bleeding. A recent study of complications associated with percutaneous biliary drains in over 450 patients demonstrated a 44% pericatheter leakage rate. Management of some pathologies requires the use of multiple drains. Current technologies and techniques are limited to draining one site from one percutaneous access. Multiple locations require multiple drains, increasing the risk of complications and worsening the patient's quality of life. Accordingly, there is interest in decreasing the duration and number of drains, and for improved external circuits to improve fluid flow and patient comfort.

### SUMMARY

The present disclosure provides, in one aspect, a percutaneous drain system (or circuit) including, an interior drain including, a catheter with a plurality of side holes formed within a side wall, a branching drain extending from the side wall through one of the side holes of the plurality of side holes, a flange disposed on the side wall, configured to secure the branching drain in position relative to the side wall, and an external circuit including, an elbow with a linear section and lock disposed at one end, a splicer disposed at an end opposite of the lock, wherein, the interior drain interfaces with the external circuit and allows for the drainage of fluid from a patient, through the interior drain and external circuit.

In some embodiments, the interior drain further comprises a radio opaque reinforced ring disposed within one of the side holes of the plurality of side holes.

In some embodiments, at least one side hole of the plurality of side holes is formed at an oblique angle to the side wall.

In some embodiments, the concept of the reinforced side hole will be used in sheaths and catheters.

In some embodiments, there will be a branching drain that can be introduced through the reinforced side hole.

In some embodiments, the flange is a pair of flanges disposed on opposite sides of the side wall.

In some embodiments, the external circuit further comprises a flange disposed between the elbow and the splicer, wherein the flange is configured to be sutured to the patient.

In some embodiments, the external circuit further comprises a cap including a pressure sensor secured to the lock.

In some embodiments, the drain itself will have a pressure sensor.

In some embodiments, the lock (three-way stop cock) is sized and configured to be part of the drain bag.

In some embodiments, the external circuit further comprises a three-way stopcock disposed on the drain bag, including a clear knob, a fixed cylinder centrally disposed within the knob, a first bar positioned perpendicular to a primary axis of the fixed cylinder, and a second bar extending from the fixed cylinder in a direction perpendicular to the primary axis and the first bar.

In some embodiments, the first bar is green in color and the second bar is red in color.

In some embodiments, the stopcock further comprises a series of LED lights.

In some embodiments, the stopcock is instead a four-way stopcock.

The present disclosure provides, in another aspect, a method for the installation of an percutaneous drain system, the method including, deploying a catheter, with a plurality of side holes formed within a side wall, within a patient, advancing a guidewire through the catheter and out one of the side holes of the plurality of side holes, extending a sheath along the guidewire and through the side hole using a pusher, advancing a branching drain along the guidewire and through the sheath, and removing the sheath, pusher and guidewire, leaving the branching drain in position.

In some embodiments, the method further comprises securing the branching drain in position relative to the side wall using a flange.

In some embodiments, the method further comprises suturing the external circuit to the patient.

In some embodiments, the method further comprises connecting the catheter to an external circuit.

In some embodiments, the method further comprises connecting a drain bag to the external circuit.

In some embodiments, the external circuit further comprises a pump including a one-way valve, in fluid connection with the drain bag, wherein the pump allows the patient pump drain fluid from the patient to the drain bag.

In some embodiments, the interior drain further comprises a pressure sensor configured to monitor pressure within the drain.

In some embodiments, the branching drain comprises a tapered tip and multiple side holes for fluid drainage.

In some embodiments, advancing the branching drain along the guidewire comprises using a pusher with a radio-opaque ring tip to position the branching drain at a desired location.

Other aspects of the disclosure will become apparent by consideration of the detailed description and accompanying drawings.

### BRIEF DESCRIPTION OF THE DRAWINGS

These and other features, aspects, and advantages of the present technology will become better understood with regards to the following figures. The accompanying figures and examples are provided by way of illustration and not by way of limitation.

FIG. 1 is a photo of a traditional biliary drain circuit.

FIG. 2 is an image of a dual drain technique with a 14 French drain and an 8 French drain within it peripherally. This method is limited as the lumen of the 14 French drain is occluded by the 8 French drain.

FIGS. 3A-3G are drawings of an exemplary branching internal drain placement of the present disclosure.

FIGS. 4A and 4B are drawings of an exemplary branching sheath/catheter of the present disclosure with angled side holes.

FIGS. 5A and 5B are drawings of an exemplary configuration of an external drain circuit of the present disclosure.

FIGS. 6A and 6B are drawings of an exemplary pressure-sensor cap of the present disclosure.

FIG. 7 is a drawing of an exemplary external drain bag of the present disclosure with a built-in three-way stopcock. It also shows a pump to help flow of fluids from the body to the drain bag.

FIGS. 8A and 8B are drawings of an exemplary three-way stopcock knob design of the present disclosure.

Before any embodiments are explained in detail, it is to be understood that the invention is not limited in its application to the details of construction and the arrangement of components set forth in the following description or illustrated in the accompanying figures. The invention is capable of other embodiments and of being practiced or of being carried out in various ways.

### DETAILED DESCRIPTION

Drains are routinely placed during interventional radiology procedures. These drains are placed into various body parts and for many different reasons. The present disclosure provides systems and methods to decrease the number of drains necessary to address areas near to the primary drainage site while decreasing the frequency of complications.

The internal components of the present disclosure resemble the branches of a tree. There is one tree trunk, but the multiple branches of the stems and roots increase the surface area to improve absorption efficiency and other functions. Similarly, there is a single primary drain, but additional internal drains can be placed through the primary drain that exit through side holes of the primary drain. This branching drain design allows for at least the following: drainage of the contralateral liver lobe/other liver segments to promote drainage from and maintain access across other ductal system(s); parallel catheter placement into the primary drain to increase the inner diameter of the systems across the stricture (ureteric or biliary) to provide prolonged dilatation; and drainage from multiple areas or loculations within an abscess.

In some embodiments, the external circuit components of the present disclosure consist of the following: splicer/elbow/disk with asymmetrical length for easier handling and capping; cap with integrated pressure sensor for monitoring pressure to help guide when to attach the drain to a bag; three-way stopcock positioned and incorporated into the drainage bag, enhancing patient comfort; a color coded knob of the three-way stopcock; and all connections and internal circuits are enlarged to minimize decrease in size of the lumens at various connections.

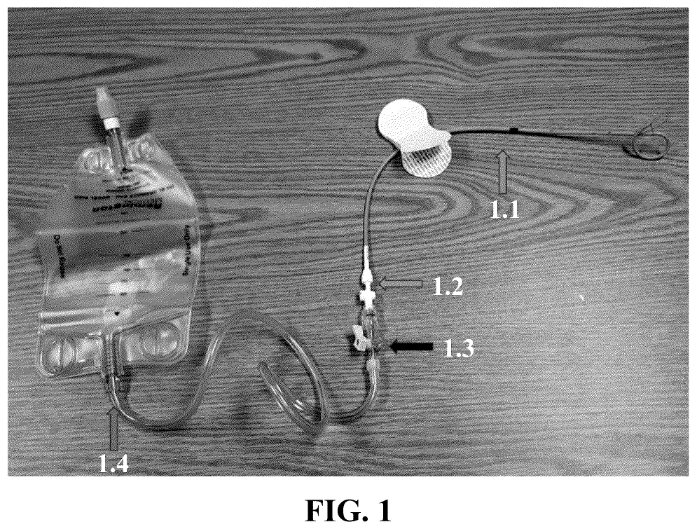

FIG. 1 is a photo of a traditional biliary drain circuit. The biliary drain (1.1) with its external end (1.2) is connected to a three-way stopcock (1.3) and drain bag (1.4). There is a significant change in luminal diameter at the various connections along the drain circuit (i.e. drain tubing to the three-way stopcock, three-way stopcock itself, and three-way stopcock to the drain bag tubing), which causes a decrease in fluid flow through the circuit. The current design of the three-way stopcock makes it difficult for operators (patients and caretakers) to easily determine which side is open and flowing. The three-way stopcock is attached in the middle of the circuit which adds bulk which leads to discomfort for patients. Current systems and techniques are also limited to draining one site from one percutaneous access. Multiple locations require multiple drains, increasing the risk of complications and worsening the quality of life.

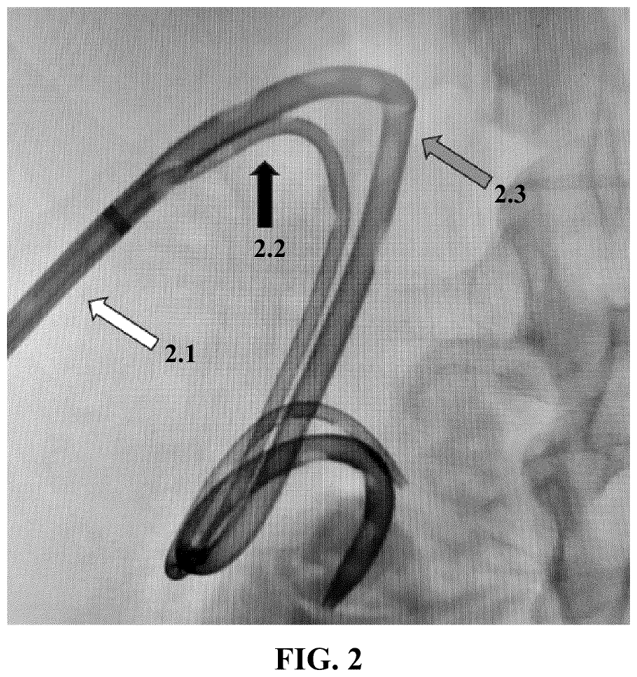

FIG. 2 is an image of a dual drain technique using a 14 French drain and a secondary 8 French drain (2.1). Before the stricture, the 8 French drain exists the first side hole (2.2) and runs parallel to the 14 French drain (2.3). This allows for the performance of at least a 22 French stricture dilatation with only a 14 French skin incision. However, this system is limited by the occlusion of the 14 French by the 8 French (as the 8 French is within the lumen of the 14 French) and the 8 French itself (as the 8 French gets kinked/constricted at the side-hole exit site).

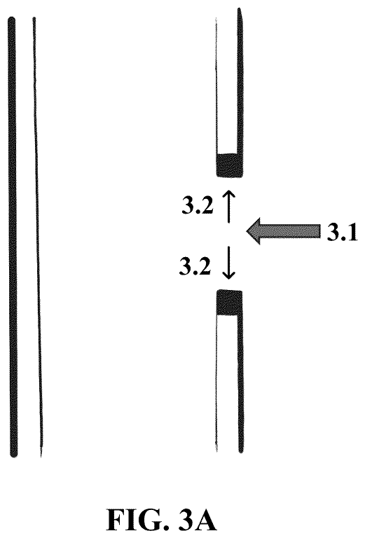

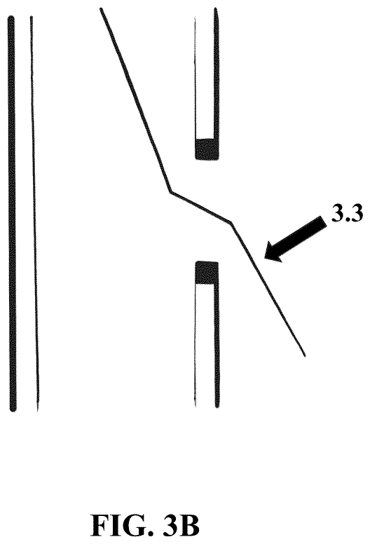

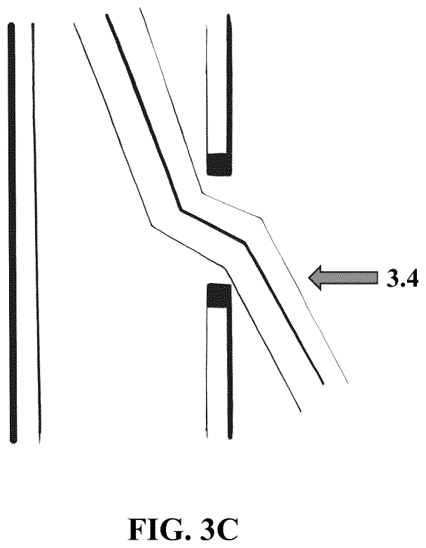

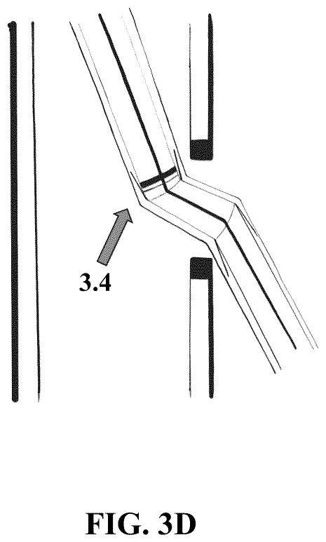

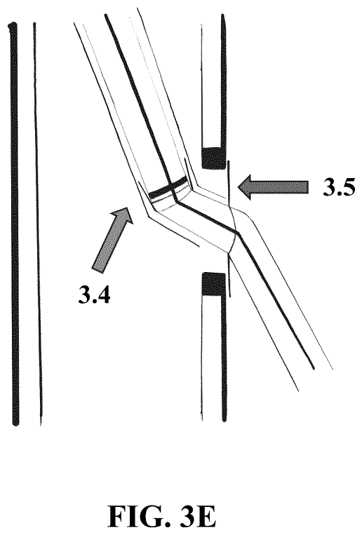

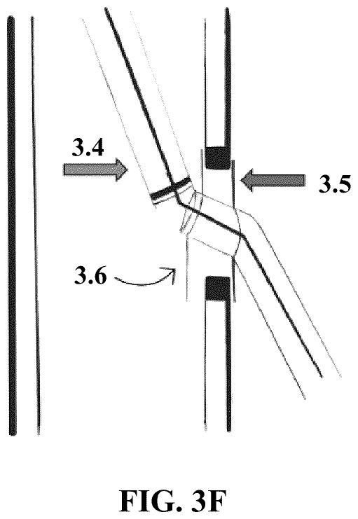

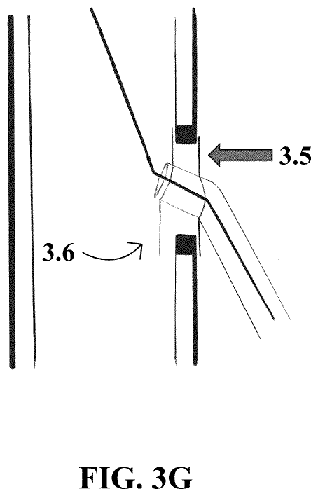

FIGS. 3A-3G are drawings of an exemplary branching internal drain placement of the present disclosure. FIG. 3A depicts a primary drain with a side hole (3.1) lined with a radio-opaque ring (3.2) to allow appropriate deployment of the branching internal drain. FIG. 3B depicts a 0.035-inch wire (3.3) being advanced into the desired location through the side hole (although wires of other dimensions may find use in embodiments herein). In some embodiments, the top several side holes are made larger than subsequent side holes. For example, a 4 mm hole allows passage of up to a 12 French (4 mm) outer diameter peel-away sheath, accommodating up to a 10 French (3.3 mm) branching internal drain. Side holes may be of any suitable sizes. In some embodiments, the side holes are lined with a radio-opaque ring to allow appropriate deployment of the branching internal drain. In some embodiments, the side holes are reinforced to allow the branching internal drain to maintain its desired angle at the exit site from the primary drain (fractal branching pattern in case of multiple branching internal drains).

FIG. 3C depicts a peel-away sheath (3.4) being advanced through the modified side hole. The side hole can be used to advance devices through sheaths and catheters where branches arise at extreme angles. This allows the disclosure to be used in all fields where catheters and sheaths, including interventional radiology, vascular surgery, and interventional cardiology. FIG. 3D depicts the branching internal drain being advanced over the wire using a pusher with a radio-opaque ring tip. FIG. 3E depicts the external flange/reinforcement (3.5) which allows the internal branching drain to maintain its desired angle and prevents inward migration. FIG. 3F depicts internal flanges (3.6) at the central tip of the branching internal drain to keep the drain in place-preventing outward movement. Arrow 3.4 in FIG. 3F points to the pusher with a radio-opaque ring tip after the sheath has been retracted. FIG. 3G depicts the branching internal drain at its desired position with the wire still in place. The presently disclosed embodiment is intended for use with percutaneous biliary drainage but may also be applied to urinary and abscess drainage.

With continued reference to the exemplary embodiment of FIGS. 3A-3G, the branching internal drain includes a wire, a sheath with dilator, a pusher, and a branching internal drain with two flanges at its peripheral tip. The 0.035-inch wire is advanced into the desired location through the appropriately positioned modified side hole. In the present embodiment, a wire with a 30-45-degree angled tip with a floppy segment (5-10 cm long) and a stiff body is used. Wires with alternative tip angles (e.g., >45 degrees, <30 degrees, etc.) and dimensions are within the scope herein. Alternatively, in some embodiments, a guidewire can be used and exchanged for a stiff working wire. In some embodiments, the sheath/dilator is advanced over the wire through the modified side hole to the desired location. In some embodiments, the dilator is then removed, leaving the wire/sheath in place. In some embodiments, the branching internal drain with multiple side holes and a tapered tip is advanced over the wire using a pusher with a radio-opaque ring tip. In some embodiments, an “external flange” keeps the branching internal drain from migrating inwards (this can occlude the lumen of the primary drain). In some embodiments, an “internal flange” keeps the branching internal drain from migrating outward, which would make subsequent retrieval difficult. In some embodiments, radio-opaque markers are positioned on the branching internal drain at the location of the flanges. In some embodiments, the sheath is pulled back to expose the “internal flange,” and the whole system is pulled back to align the “external flange” to the external portion of the modified side hole of the primary drain. In some embodiments, the sheath is retracted to expose the “internal flange” inside the drain. In some embodiments, the sheath is then removed, followed by the pusher and the wire, leaving the branching internal drain in place. In some embodiments, the subsequent increased total diameter of the internal component of the drain helps secure its position inside the patient and decrease dislodgement.

An exemplary removal mechanism of branching internal drain includes a wire and a balloon for retrieval. In such embodiments, the wire is advanced through the ostium of the branching internal drain. A small non-compliant balloon is then advanced over the wire into the branching internal drain. The balloon is then inflated within the stent (branching internal drain). The balloon, now holding the branching internal drain from the inside, is retracted into the primary drain, folding it while keeping the wire in position. The system is removed while maintaining wire access. Alternative removal processes are within the scope herein.

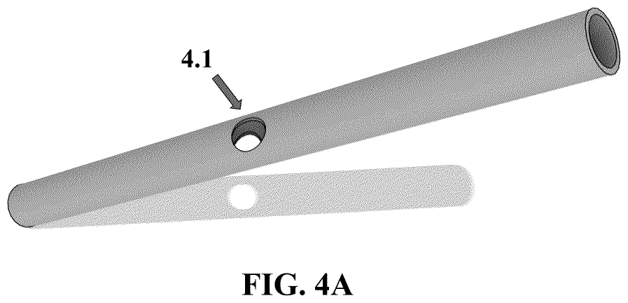

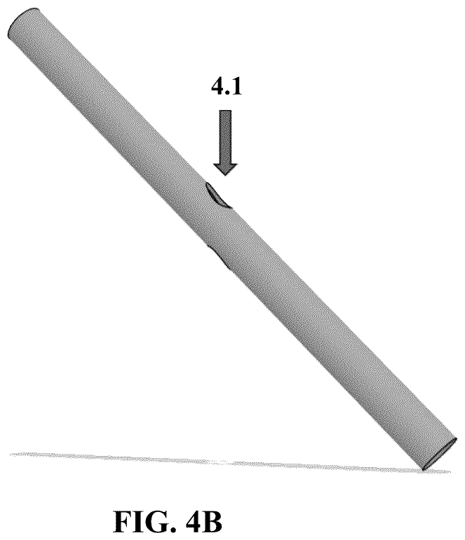

FIGS. 4A and 4B are drawings of an exemplary branching sheath/catheter of the present disclosure with angled side holes (4.1). In some embodiments, the side hole angled upwards could be used to access branches that are at an acute angle. In some embodiments, the side hole angled downwards could be used to access branches that are at an obtuse angle.

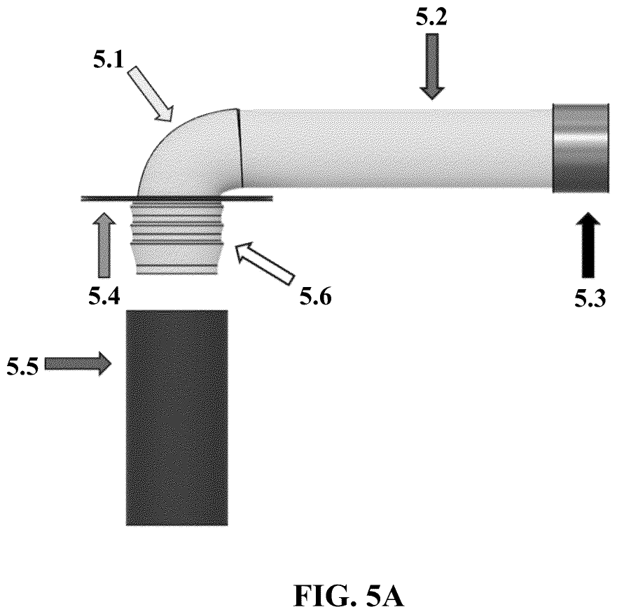

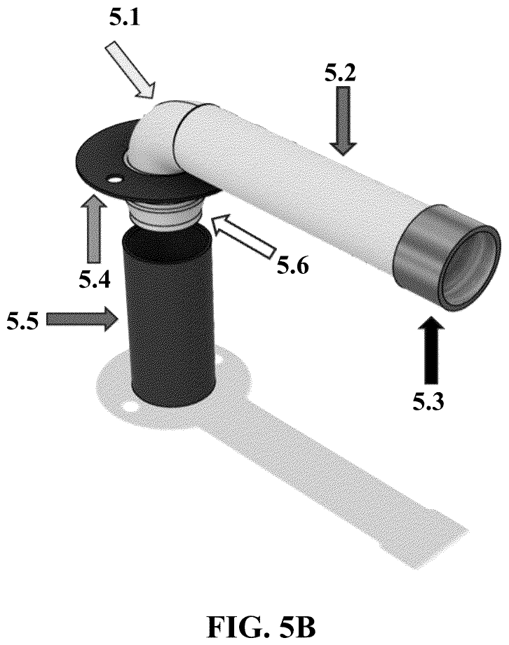

FIGS. 5A and 5B are drawings of an exemplary configuration of the external drain circuit of the present disclosure. Elbow connector (5.1) with a long outer tube (5.2) with a Luer lock (5.3). There is a circular flange, or disk, (5.4) with holes to help suture the elbow to the skin to prevent dislodgement. The drain end (5.5) is joined to the splicer (5.6).

In some embodiments, the elbow includes two distinct ends. The 90-degree angle of the elbow provides a barrier against the inward displacement of the drain. In some embodiments, the angle of the elbow is less than 90 degrees (e.g., 85, 80, 75, 70, or less). In some embodiments, the angle of the elbow is greater than 90 degrees (e.g., 95, 100, 105, 110, 120, or more). In some embodiments, the disc allows suturing of the elbow component to the skin, decreasing the chance of drain dislodgement. In some embodiments, the first end (splicer) connects to the part of the drain that exits the skin. This allows the diameter to be slightly larger than the drain, minimizing leakage. This also allows for the diameter of the drain and the connector to not decrease at the connection. In some embodiments, the second end is configured to attach to the drainage bag tube. In some embodiments, the end connected to the drainage bag tube is longer than the end attached to the skin-exiting drain. The longer length of the elbow away from the skin exit site would allow patients to comfortably detach the drain bag and cap the elbow without the hindrance of limited space, enhancing manual dexterity and ease of operation.

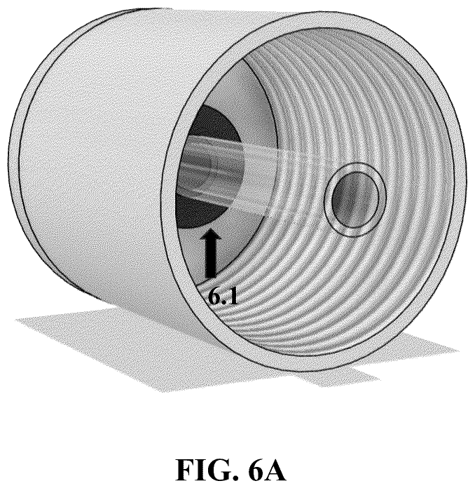

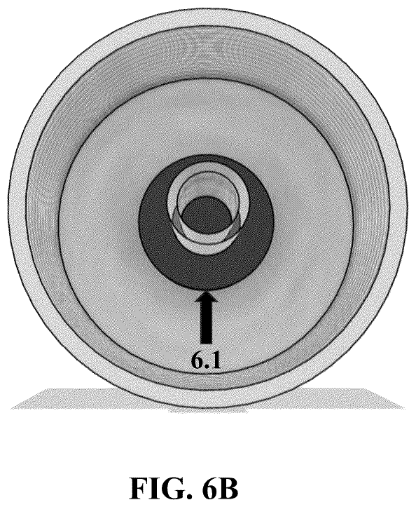

FIGS. 6A and 6B are drawings of an exemplary pressure-sensor cap of the present disclosure. The inner surface of the drain cap (6.1) houses a pressure sensor. In some embodiments, pressure sensor (manometer) is built into the end of the elbow where the cap is applied. In some embodiments, pressure sensor (manometer) is built into the drain itself. This sensor alerts patients to the optimal time to remove the cap and reattach the drainage bag tube. Inclusion of the pressure sensor enhances patient comfort by enabling them to know when to detach/reattach the drainage bag. All drains do not need to be always attached to a drain bag. Not having a bulky drain bag while patients are doing activities such as showering and sleeping has a significant impact on the quality of life of these patients. This is particularly beneficial as carrying the drainage bag all the time can be cumbersome. In some embodiments, the sensor alerts patients (via a light/sound or a prompt via a connection to the phone) to increase pressure, indicating the need to reattach the drainage bag. This feature allows patients to enjoy more freedom and comfort in their daily activities, only reattaching the bag, when necessary, based on the sensor's indication.

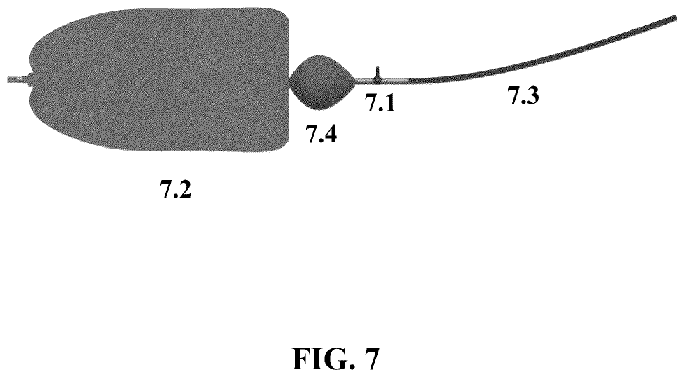

FIG. 7. is a drawing of an exemplary external drain bag of the present disclosure with a built-in three-way stopcock. In some embodiments, a built-in three-way stopcock (7.1) is incorporated into the bag (7.2). The bulb (7.4) is a manual pump with a one-way valve to help flow of fluids from the body to the bag, independent of gravitational forces. The tubing (7.3) has a Luer lock that connects to the tubing coming from the elbow tubing. In the embodiment depicted in FIG. 7, the stopcock is positioned at the end of the drainage bag to improve patient comfort by moving it away from the patient's skin. Stopcocks can be a source of discomfort, mainly when patients are lying down or moving.

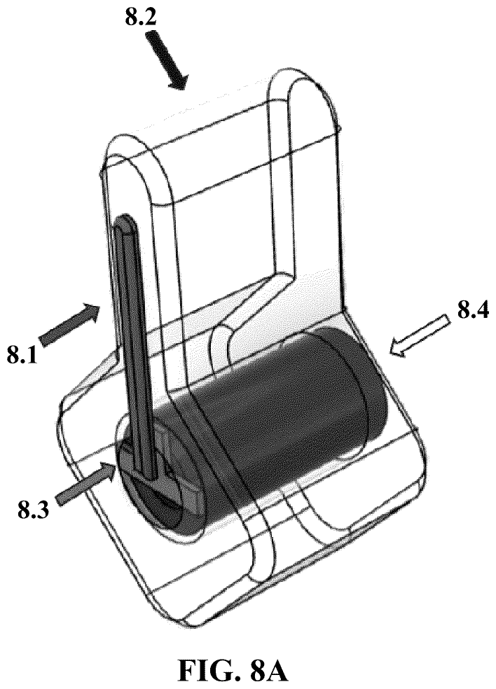

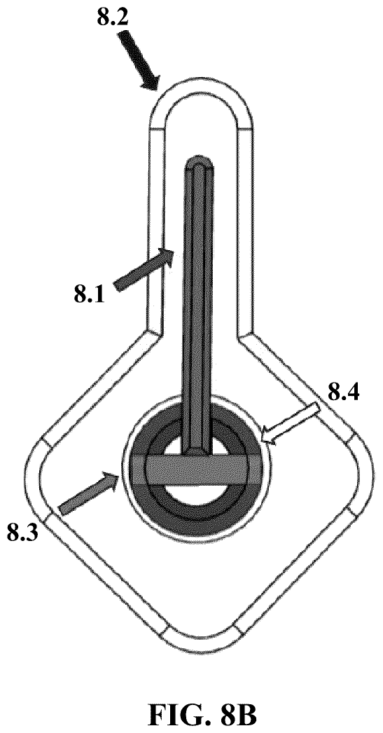

FIGS. 8A and 8B are drawings of an exemplary three-way stopcock knob design of the present disclosure. Arrow 8.1 points to red bar on the clear knob (8.2) which covers the green line (8.3) (symbolizing closed channel) on the fixed component (8.4).

Traditional three-way stopcocks use an “OFF” arrow to indicate the closed port, often leading to operator's confusion regarding the fluid flow direction. Some embodiments herein employ a color-coding scheme inspired by universal traffic light systems to indicate each port's functional status clearly. In some embodiments, the transparent knob of the three-way stopcock features a red bar to distinctly mark the “OFF” position. In some embodiments, this red indicator points toward the port that is currently closed, while covering the internal green bar signifying that that limb is occluded. Red, associated with ‘stop’ in traffic signals, serves as an immediate visual cue that the flow of fluids through this port is blocked. In some embodiments, the fixed cylinder/disk that the knob rotates on has green bars. In some embodiments, the red bar from the knob above covers the first green bar. The other two green bars provides a visual cue indicating the flow of fluid through these pathways. Green is commonly associated with ‘go’ or ‘active’ conditions, thus providing a clear contrast to the red ‘stop indicator. In some embodiments, the green lines could be light emitting diodes (LED's) to allow visualization of the open channels in the dark. This color-coding system allows for a quick and accurate assessment of which ports are open and closed and reduces the likelihood of misinterpretation. By aligning the design with universally recognized color associations, the stopcock becomes more user-friendly. In some embodiments, this three-way stopcock is expanded to become a 4-way or higher port “portal” so two or more drains can be connected to one external bag. This 4-way allows closure of one channel, while allowing flow through the two drains into the bag or the flush channel towards both drains. While particularly beneficial in the context of the present disclosure, this enhanced three-way stopcock design has potential applications where such connectors are used. For example, many venous and arterial lines in the intensive care unit.

The drain and circuit of the present disclosure may be used in a number of non-limiting applications: drainage of the contralateral liver lobe/other liver segments to promote drainage from and maintain access across other ductal system(s); parallel catheter placement into the primary drain to increase the inner diameter of the systems across the stricture (ureteric or biliary) to provide prolonged dilatation; drainage from multiple areas or loculations within an abscess; the side holes in drains, catheters, and sheaths can be used to advance devices into branches that originate at a right angle or acute/obtuse angle; increasing patient comfort and ease of use through the use of a color-coded stopcock positioned away from the patient's skin; increase fluid flow due to the lack of sudden luminal diameter decreases through the drain and circuit.

The drain and circuit of the present disclosure has a number of non-limiting advantages over current technologies, including allowing for multiple drainage sites using the same access incision; maintaining access to multiple drainage sites, providing potential dilatation of the stricture with multiple drains internally, improved quality of life and patient comfort by minimizing bulky external component by using a drain cap with a manometer and by integrating the three-way stopcock with drain bag; decreasing complications from multiple drains; providing improved flow due to increased luminal diameter across the circuit; and improved usability through the use of color coding.

Various features and advantages are set forth in the following claims.

### REFERENCES

All publications, patent applications, patents, and other references identified herein are incorporated by reference in their entirety.
Devane A M, Annam A, Brody L, et al. Society of Interventional Radiology Quality Improvement Standards for Percutaneous Cholecystostomy and Percutaneous Transhepatic Biliary Interventions. J Vasc Interv Radiol. November 2020; 31 (11): 1849-1856. Doi:10.1016/j.jvir.2020.07.015.
Nennstiel S, Weber A, Frick G, et al. Drainage-related Complications in Percutaneous Transhepatic Biliary Drainage: An Analysis Over 10 Years. J Clin Gastroenterol. October 2015; 49 (9): 764-70. Doi:10.1097/mcg.0000000000000275.
Gwon D I, Sung K B, Ko G Y, Yoon H K, Lee S G. Dual catheter placement technique for treatment of biliary anastomotic strictures after liver transplantation. Liver Transpl. February 2011; 17 (2): 159-66. Doi:10.1002/lt.22206.
Adverse Events after Percutaneous Transhepatic Biliary Drainage: A Ten-Year Retrospective Analysis, Matthew Antalek, Muhammed E Patel, Gabriel M Knight, Asad Malik, Ali Husnain, Kristine Stiff, Abhinav Talwar, Allison Reiland, Albert Nemcek Jr, Riad Salem, Ahsun Riaz J Vasc Interv Radiol. 2024 Dec. 24: S1051-0443 (24) 00810-8. doi: 10.1016/j.jvir.2024.12.022.

What is claimed is:

1. A percutaneous drain system comprising:
an interior drain including,
a catheter with a plurality of side holes formed within a side wall;
a branching drain extending from the side wall through one of the side holes of the plurality of side holes;
a flange disposed on the side wall, configured to secure the branching drain in position relative to the side wall; and
an external circuit including,
an elbow with a linear section and lock disposed at one end;
a splicer disposed at an end opposite of the lock;
wherein, the interior drain interfaces with the external circuit and allows for the drainage of fluid from a patient, through the interior drain and external circuit.

2. The system of claim 1, wherein the interior drain further comprises a radio opaque ring disposed within one of the side holes of the plurality of side holes.

3. The system of claim 1, wherein the flange is a pair of flanges disposed on opposite sides of the side wall.

4. The system of claim 1, wherein the lock is sized and configured to connect to a drain bag.

5. The system of claim 1, wherein the external circuit further comprises a flange disposed between the elbow and the splicer, wherein the flange is configured to be sutured to the patient's skin.

6. The system of claim 1, wherein at least one side hole of the plurality of side holes is formed at an oblique angle to the side wall.

7. The system of claim 1, wherein the external circuit further comprises a cap including a pressure sensor secured to the lock.

8. The system of claim 4, wherein the external circuit further comprises a three-way stopcock disposed on the drain bag, comprising:
a clear knob;
a fixed cylinder centrally disposed within the knob;
a first bar positioned perpendicular to a primary axis of the fixed cylinder; and
a second bar extending from the fixed cylinder in a direction perpendicular to the primary axis and the first bar.

9. The system of claim 8, wherein the first bar is green in color and the second bar is red in color.

10. The system of claim 8, wherein the stopcock further comprises a series of LED lights.

11. The system of claim 8, wherein the stopcock is instead a four-way stopcock.

12. A method for the installation of a percutaneous drain system, the method comprising:
deploying a catheter, with a plurality of side holes formed within a side wall, within a patient;
advancing a guidewire through the catheter and out one of the side holes of the plurality of side holes;
extending a sheath along the guidewire and through the side hole using a pusher;
advancing a branching drain along the guidewire and through the sheath; and
removing the sheath, pusher and guidewire, leaving the branching drain in position.

13. The method of claim 12, wherein the method further comprises securing the branching drain in position relative to the side wall using a flange.

14. The method of claim 12, wherein the method further comprises suturing the external circuit to the patient.

15. The method of claim 12, wherein the method further comprises connecting the catheter to an external circuit.

16. The method of claim 15, wherein the method further comprises connecting a drain bag to the external circuit.

17. The drain system of claim 4, wherein the external circuit further comprises a pump including a one-way valve, in fluid connection with the drain bag, wherein the pump allows the patient pump drain fluid from the patient to the drain bag.

18. The system of claim 1, wherein the interior drain further comprises a pressure sensor configured to monitor pressure within the drain.

19. The system of claim 1, wherein the branching drain comprises a tapered tip and multiple side holes for fluid drainage.

20. The system of claim 12, wherein advancing the branching drain along the guidewire comprises using a pusher with a radio-opaque ring tip to position the branching drain at a desired location.
